(19) United States
(12) Patent Application Publication
(10) Pub. No.: US 2020/0038852 A1
(43) Pub. Date: Feb. 6, 2020

(54) UNSATURATED HYDROCARBON PRODUCTION METHOD AND DEHYDROGENATION CATALYST REGENERATION METHOD

(71) Applicant: JXTG Nippon Oil & Energy Corporation, Tokyo (JP)

(72) Inventors: Nobuhiro KIMURA, Tokyo (JP); Atsushi SEGAWA, Tokyo (JP); Tatsuya ICHIJO, Tokyo (JP)

(73) Assignee: JXTG Nippon Oil & Energy Corporation, Tokyo (JP)

(21) Appl. No.: 16/606,018

(22) PCT Filed: Dec. 27, 2017
(86) PCT No.: PCT/JP2017/046968

§ 371 Date: Oct. 17, 2019

(30) Foreign Application Priority Data
Apr. 21, 2017 (JP) 2017-084567

(51) Int. Cl. B01J 38/12; B01J 23/62; B01J 23/96; B01J 37/02; B01J 37/08; C07C 5/333

(52) U.S. Cl. CPC B01J 38/12; B01J 23/626; B01J 23/96; B01J 37/0201; C07C 2523/42; B01J 37/08; C07C 5/3337; C07C 2523/14; B01J 37/0236

(57) ABSTRACT

A method for producing an unsaturated hydrocarbon comprising: a dehydrogenation step of contacting a raw material gas containing at least one hydrocarbon selected from a group consisting of alkanes and olefins with a dehydrogenation catalyst containing a group 14 metal element and Pt to obtain a product gas containing at least one unsaturated hydrocarbon selected from the group consisting of olefins and conjugated dienes, and a regeneration step of contacting the dehydrogenation catalyst subjected to the dehydrogenation step with a regenerating gas containing molecular oxygen under a temperature condition of 310 to 450° C.

### TECHNICAL FIELD

The present invention relates to a method for producing an unsaturated hydrocarbon and a method for regenerating a dehydrogenation catalyst.

### BACKGROUND ART

An increase in the demand of conjugated dienes including butadiene as a raw material for synthetic rubbers, or the like has been anticipated because of motorization centering on Asia in recent years.

For example, a method for producing a conjugated diene by a direct dehydrogenation reaction of n-butane using a dehydrogenation catalyst (Patent Literature 1) and methods for producing a conjugated diene by an oxidative dehydrogenation reaction of n-butene (Patent Literatures 2 to 4) have been known as methods for producing conjugated diene.

### CITATION LIST

### Patent Literature

Patent Literature 1 Japanese Unexamined Patent Publication No. 2014-205135

Patent Literature 2 Japanese Unexamined Patent Publication No. S57-140730

Patent Literature 3 Japanese Unexamined Patent Publication No. S60-1139

Patent Literature 4 Japanese Unexamined Patent Publication No. 2003-220335

### SUMMARY OF INVENTION

### Technical Problem

Along with an increase in the demand of unsaturated hydrocarbons, development of various methods for producing unsaturated hydrocarbons is required, the methods differing in features such as required properties, operating cost, and reaction efficiency of a producing device.

With a method for obtaining an unsaturated hydrocarbon by means of a dehydrogenation reaction of an alkane or olefin, coke is deposited onto a catalyst used for the dehydrogenation reaction, and the reaction efficiency gradually decreases. Catalyst regeneration is performed regularly by means of firing or the like in order to remove this coke, but in the catalyst after the regeneration, the catalyst activity may markedly decrease.

It is an object of the present invention to provide a method for regenerating a dehydrogenation catalyst, wherein coke deposited on a dehydrogenation catalyst can be efficiently removed while the catalyst activity of the dehydrogenation catalyst is sufficiently maintained. Additionally, it is another object of the present invention to provide a method for producing an unsaturated hydrocarbon, wherein efficiency improvement of an entire process is achieved by efficiently removing coke deposited on a dehydrogenation catalyst while the catalyst activity of the dehydrogenation catalyst is sufficiently maintained.

### Solution to Problem

An aspect of the present invention relates to a method for producing an unsaturated hydrocarbon comprising a dehydrogenation step of contacting a raw material gas containing at least one hydrocarbon selected from the group consisting of alkanes and olefins with a dehydrogenation catalyst containing a group 14 metal element and Pt to obtain a product gas containing at least one unsaturated hydrocarbon selected from the group consisting of olefins and conjugated dienes, and a regeneration step of contacting the dehydrogenation catalyst subjected to the dehydrogenation step with a regenerating gas containing molecular oxygen under a temperature condition of 310 to 450° C.

With the above production method, it is possible to efficiently remove coke deposited on the dehydrogenation catalyst while sufficiently maintaining the catalyst activity of the dehydrogenation catalyst, by regenerating the dehydrogenation catalyst subjected to the dehydrogenation step under particular conditions. That is, with the above production method, it is possible to accomplish efficiency improvement of the entire process because a dehydrogenation catalyst having high catalyst activity can be recovered while an unsaturated hydrocarbon is obtained.

In one aspect, the dehydrogenation catalyst may include Sn as a group 14 metal element.

In one aspect, the dehydrogenation catalyst may be a catalyst in which a group 14 metal element and Pt are supported on a carrier using a metal source containing no chlorine atom.

In one aspect, the raw material gas may contain an alkane having 2 to 10 carbon atoms.

In one aspect, the raw material gas may contain an olefin having 4 to 10 carbon atoms.

Another aspect of the present invention relates to a method of regenerating a dehydrogenation catalyst containing a group 14 metal element and Pt that has been used for a dehydrogenation reaction of a hydrocarbon, the method comprising a regeneration step of contacting the dehydrogenation catalyst with a regenerating gas containing molecular oxygen under a temperature condition of 310 to 450° C.

According to the above regeneration method, it is possible to efficiently remove coke deposited on the dehydrogenation catalyst while sufficiently maintaining the catalyst activity of the dehydrogenation catalyst.

Still another aspect of the present invention relates to a method for producing an unsaturated hydrocarbon comprising a step of performing a dehydrogenation reaction of an alkane using a dehydrogenation catalyst regenerated by the above regeneration method to obtain at least one unsaturated hydrocarbon selected from the group consisting of olefins and conjugated dienes.

Even another aspect of the present invention relates to a method for producing an unsaturated hydrocarbon comprising a step of performing a dehydrogenation reaction of an olefin using a dehydrogenation catalyst regenerated by the above regeneration method to obtain a conjugated diene.

### Advantageous Effects of Invention

According to the present invention, provided is a method for regenerating a dehydrogenation catalyst, wherein coke deposited on a dehydrogenation catalyst can be efficiently removed while the catalyst activity of the dehydrogenation catalyst is sufficiently maintained. Additionally, according to the present invention, provided is a method for producing an unsaturated hydrocarbon, wherein efficiency improvement of the entire process can be achieved by efficiently removing coke deposited on the dehydrogenation catalyst while the catalyst activity of the dehydrogenation catalyst is sufficiently maintained.

### DESCRIPTION OF EMBODIMENTS

Hereinbelow, one suitable embodiment of the present invention will be described.

A method for producing a dehydrogenation catalyst according to the present invention comprises a dehydrogenation step of contacting a raw material gas containing at least one hydrocarbon selected from the group consisting of alkanes and olefins with a dehydrogenation catalyst containing a group 14 metal element and Pt to obtain a product gas containing at least one unsaturated hydrocarbon selected from the group consisting of olefins and conjugated dienes, and a regeneration step of contacting the dehydrogenation catalyst subjected to the dehydrogenation step with a regenerating gas containing molecular oxygen under a temperature condition of 310 to 450° C.

With the production method of the present embodiment, the used dehydrogenation catalyst can be efficiently regenerated in the regeneration step while an unsaturated hydrocarbon is obtained in the dehydrogenation step. The dehydrogenation catalyst regenerated in the regeneration step may be reused in the dehydrogenation step or may be utilized in other steps.

In the present embodiment, the raw material gas contains at least one hydrocarbon selected from the group consisting of alkanes and olefins. When the raw material gas contains an alkane, the dehydrogenation step may be a step of obtaining at least one unsaturated hydrocarbon selected from the group consisting of olefins and conjugated dienes by means of a dehydrogenation reaction of the alkane. Alternatively, when the raw material gas contains an olefin, the dehydrogenation step may be a step of obtaining a conjugated diene by means of a dehydrogenation reaction of the olefin. The raw material gas may be one that contains either one of an alkane or an olefin or may be one that contains both of an alkane and an olefin.

The number of carbon atoms in the hydrocarbon contained in the raw material gas may be the same as the number of carbon atoms of an intended unsaturated hydrocarbon. The number of carbon atoms of the alkane, for example, may be 2 or more, may be 3 or more, or may be 4 or more. Alternatively, the number of carbon atoms of the alkane, for example, may be 10 or less or may be 6 or less. The number of carbon atoms of the olefin is only required to be 4 or more. Alternatively, the number of carbon atoms of the olefin, for example, may be 10 or less or may be 6 or less.

The alkane may be, for example, chain-like, or may be cyclic. Examples of the chain-like alkane include butane, pentane, hexane, heptane, octane, and decane. More specific examples of the linear alkane include n-butane, n-pentane, n-hexane, n-heptane, n-octane, and n-decane. Additionally, examples of the branched alkane include isobutane, isopentane, 2-methylpentane, 3-methylpentane, 2,3-dimethylpentane, isoheptane, isooctane, and isodecane. Examples of the cyclic alkane include cyclopentane, cyclohexane, cycloheptane, cyclooctane, cyclodecane, and methylcyclohexane. The raw material gas may contain one alkane or may contain two or more alkanes.

The olefin, for example, may be chain-like, or may be cyclic. A chain-like olefin may be at least one selected from the group consisting of, for example, butene, pentene, hexene, heptene, octene, nonene, and decene. The chain-like olefin may be linear or may be branched. The linear olefin may be at least one selected from the group consisting of, for example, n-butene, n-pentene, n-hexene, n-heptene, n-octene, n-nonene, and n-decene. The branched olefin may be at least one selected from the group consisting of, for example, isopentene, 2-methylpentene, 3-methylpentene, 2,3-dimethylpentene, isoheptene, isooctene, isononene, and isodecene. The raw material gas may be one that contains one of the olefins singly or may be one that contains two or more of these.

In the raw material gas, the partial pressure of the hydrocarbon may be 1.0 MPa or less, may be 0.1 MPa or less, or may be 0.01 MPa or less. The conversion rate of the hydrocarbon is more likely to be enhanced by lowering the hydrocarbon partial pressure of the raw material gas.

Also, the partial pressure of the hydrocarbon in the raw material gas is preferably 0.001 MPa or more, more preferably 0.005 MPa or more, from the viewpoint of reducing the size of a reactor with respect to a raw material flow rate.

The raw material gas may further contain an inert gas such as nitrogen or argon. Also, the raw material gas may further contain steam.

When the raw material gas contains steam, the content of the steam is preferably 1.0 times moles or more, more preferably 1.5 times moles or more, with respect to the hydrocarbon. Deterioration in the activity of the catalyst may be suppressed by incorporation of steam in the raw material gas. The content of the steam, for example, may be 50 times moles or less and is preferably 10 times moles or less with respect to the hydrocarbon.

The raw material gas may further contain other components such as hydrogen, oxygen, carbon monoxide, carbon dioxide, and dienes in addition to the above.

In the present embodiment, the product gas contains at least one unsaturated hydrocarbon selected from the group consisting of olefins and conjugated dienes. The number of carbon atoms of each of the olefin and the conjugated diene may be the same as the number of carbon atoms of the hydrocarbon in the raw material gas. For example, the number of carbon atoms of the olefin contained in the product gas may be 2 or more, may be 3 or more, or may be 4 or more. Additionally, the number of carbon atoms of the olefin contained in the product gas, for example, may be 10 or less or may be 6 or less. Also, the number of carbon atoms of the conjugated diene contained in the product gas, for example, may be 4 to 10 or may be 4 to 6.

Examples of the olefin include ethylene, propylene, butene, pentene, hexene, heptene, octene, nonene, and decene, and these may be any isomers. Examples of the conjugated diene include 1,3-butadiene, 1,3-pentadiene, isoprene, 1,3-hexadiene, 1,3-heptadiene, 1,3-octadiene, 1,3-nonadiene, and 1,3-decadiene. The product gas may be one that contains one unsaturated hydrocarbon or may be one that contains two or more unsaturated hydrocarbons. For example, the product gas may contain an olefin and a conjugated diene.

Hereinbelow, the dehydrogenation catalyst in the present embodiment will be described in detail.

The dehydrogenation catalyst is a solid catalyst containing a group 14 metal element and Pt. The dehydrogenation catalyst may be a catalyst in which a supported metal containing a group 14 metal element and Pt, for example, is supported on a carrier.

It is preferred that the carrier be an inorganic oxide carrier. Examples of the inorganic oxide carrier include supports containing an inorganic oxide such as alumina, alumina magnesia, magnesia, titania, silica, silica alumina, silica magnesia, ferrite, and spinel structures (magnesium spinel, iron spinel, zinc spinel, manganese spinel).

As the carrier, a carrier containing aluminum (Al) is preferred. The content of Al in the carrier may be 25% by mass or more, and is preferably 50% by mass or more, based on the total mass of the carrier.

It is preferred that the carrier be a spinel structural body having a spinel structure, for example, magnesium spinel (MgAl₂O₄). Thereby, the acidity of the carrier is lowered, and an effect of suppressing carbon precipitation is exerted.

On the dehydrogenation catalyst, a supported metal containing a group 14 metal element and Pt is supported. The group 14 metal element may be at least one selected from the group consisting of Ge, Sn, and Pb, and is preferably Sn.

The dehydrogenation catalyst can be suitably utilized as a catalyst for a dehydrogenation reaction of an alkane as a raw material. In this dehydrogenation reaction, at least one unsaturated hydrocarbon selected from the group consisting of olefins and conjugated diener is obtained from the alkane.

The dehydrogenation catalyst can be also suitably utilized as a catalyst for a dehydrogenation reaction of an olefin as a raw material. In this dehydrogenation reaction, a conjugated diene is obtained from the olefin.

When the catalyst is for a dehydrogenation reaction of an alkane as a raw material, the amount of the group 14 metal element supported in the dehydrogenation catalyst, for example, may be 1% by mass or more, is preferably 1.3% by mass or more, and may be 9% by mass or less, is preferably 7% by mass or less, based on the total mass of the dehydrogenation catalyst. With such an amount supported, dehydrogenation performance in a dehydrogenation reaction of an alkane as a raw material is enhanced as well as precipitation of coke in the dehydrogenation reaction is suppressed to enable the service life of the catalyst to be extended.

When the catalyst is for a dehydrogenation reaction of an olefin as a raw material, the amount of the group 14 metal element supported in the dehydrogenation catalyst, for example, may be 5% by mass or more, is preferably 7% by mass or more, and may be 25% by mass or less, preferably 18% by mass or less, based on the total mass of the dehydrogenation catalyst. With such an amount supported, dehydrogenation performance in a dehydrogenation reaction of an olefin as a raw material is enhanced as well as precipitation of coke in the dehydrogenation reaction is suppressed to enable the service life of the catalyst to be extended.

The amount of Pt supported in the dehydrogenation catalyst, for example, may be 0.1% by mass or more, is preferably 0.5% by mass or more, based on the total mass of the dehydrogenation catalyst. Thereby, the catalyst activity of the dehydrogenation catalyst is further enhanced. Additionally, the amount of Pt supported in the dehydrogenation catalyst, for example, may be 5% by mass or less, is preferably 2% by mass or less, based on the total mass of the dehydrogenation catalyst. Thereby, the dispersibility of Pt in the dehydrogenation catalyst is enhanced, and the activity per amount supported tends to be enhanced.

Note that the amount of each of the group 14 metal element and Pt supported in the dehydrogenation catalyst can be measured by emission spectroscopic analysis using a high-frequency inductively coupled plasma (ICP) as a light source. In the measurement, a sample solution is atomized and introduced into Ar plasma, the light emitted when excited element returns to the ground state is spectrally separated, the element is qualified by means of its wavelength, and the element is quantified by means of its intensity.

In the dehydrogenation catalyst, the group 14 metal element and Pt may interact with each other, for example, may form an alloy. Thereby, the durability of the dehydrogenation catalyst tends to be enhanced.

The dehydrogenation catalyst can be suitably utilized as a catalyst for a dehydrogenation reaction of an alkane as a raw material or as a catalyst for a dehydrogenation reaction of an olefin as a raw material. Note that the dehydrogenation catalyst can be used also in applications other than these and can be used as a catalyst for a dehydrogenation reaction of an oxygen-containing compound such as alcohol, aldehyde, ketone, and carboxylic acid, a catalyst for a hydrogenation reaction, which is a reverse reaction of the dehydrogenation reaction, or the like. In a regeneration step mentioned below, a regenerated dehydrogenation catalyst may be applied to any of these reactions.

The dehydrogenation catalyst may be used for the reaction after subjected to a reduction treatment. The reduction treatment can be, for example perfoimed by retaining the dehydrogenation catalyst under reducing gas atmosphere at 40 to 600° C. The retention time may be, for example, 0.05 to 24 hours. The reducing gas may be, for example, hydrogen or carbon monoxide.

The dehydrogenation catalyst may further contain a molding auxiliary agent from the viewpoint of improving moldability. The molding auxiliary agent may be, for example, a thickener, surfactant, humectant, plasticizer, or binder raw material.

The shape of the dehydrogenation catalyst is not particularly limited and may be, for example, a shape such as a pellet shape, granular shape, honeycomb shape, or sponge shape.

In connection with the method for producing a dehydrogenation catalyst, examples of a method for supporting the supported metal on the carrier, include, but not particularly limited to, an impregnation method, precipitation method, coprecipitation method, kneading method, ionic exchange method, and pore-filling method.

In the present embodiment, for example, after the group 14 metal element is supported on the carrier, Pt may be supported. Alternatively, after Pt is supported on the carrier, the group 14 metal element may be supported. Alternatively, the group 14 metal element and Pt may be supported simultaneously on the carrier.

An example of the method for supporting a supported metal on a carrier includes a method in which a solution prepared by dissolving a metal source containing a supported metal is provided, a carrier is impregnated with this solution, dried, and fired to support the group 14 metal element on the carrier.

The metal source containing the supported metal may be, for example, a metal salt or complex. The metal salt of the supported metal may be, for example, an inorganic salt, organic acid salt, or hydrate thereof. The inorganic salt may be, for example, a sulfate, nitrate, chloride, phosphate, or carbonate. The organic acid salt may, for example, be an acetate or succinate. Alternatively, the complex of the supported metal may be, for example, an alkoxide complex or ammine complex.

As the metal source, a metal source containing no chlorine atom is suitably used. With a dehydrogenation catalyst in which a group 14 metal element and Pt are supported on a carrier using a metal source containing no chlorine atom, reduction in the catalyst activity in the regeneration step tends to be markedly suppressed.

Examples of a metal source containing no chlorine atom and containing a group 14 metal element include sodium stannate, potassium stannate, tin sulfate, tin oxide, tin nitrate, tin acetate, metastannic acid, and tin chloride.

Alternatively, examples of a metal source containing no chlorine atom and containing Pt include tetraammineplatinic(II) acid, tetraammineplatinic(II) acid salt (e.g., nitrate), tetraammineplatinic(II) acid hydroxide solution, dinitrodiammineplatinum(II) nitric acid solution, hexahydroxoplatinic(IV) acid nitric acid solution, and hexahydroxoplatinic(IV) acid ethanolamine solution.

Next, the dehydrogenation step in the present embodiment will be described in detail.

The dehydrogenation step is a step of performing a dehydrogenation reaction of a hydrocarbon by contacting a raw material gas with a dehydrogenation catalyst to obtain a product gas containing an unsaturated hydrocarbon of hydrocarbon.

The dehydrogenation step may be, for example, performed using a reactor filled with the dehydrogenation catalyst, by circulating the raw material gas through the reactor. As the reactor, various reactors used for a gas phase reaction by use of a solid catalyst can be used. Examples of the reactor include a fixed-bed insulation type reactor, radial flow type reactor, and tube-type reactor.

The reaction form of the dehydrogenation reaction may be, for example, a fixed-bed type, moving-bed type, or fluidized-bed type. Among these, a fixed-bed type is preferred from the viewpoint of equipment cost.

The reaction temperature of the dehydrogenation reaction, that is, the temperature in the reactor may be 300 to 800° C., or may be 500 to 700° C., from the viewpoint of the reaction efficiency. When the reaction temperature is 500° C. or more, the amount of an unsaturated hydrocarbon to be generated tends to further increase. When the reaction temperature is 700° C. or less, the high activity tends to be maintained over a longer period of time.

The reaction pressure, that is, the atmospheric pressure in the reactor may be 0.01 to 1 MPa, may be 0.05 to 0.8 MPa, or may be 0.1 to 0.5 MPa. When the reaction pressure is in the above range, the dehydrogenation reaction is likely to proceed, and more excellent reaction efficiency tends to be obtained.

When the dehydrogenation step is performed in a continuous reaction form for continuously supplying a raw material gas, a weight hourly space velocity (hereinbelow, it is referred to as “WHSV”) may be 0.1 h⁻¹ or more or may be 1.0 h⁻¹ or more, and may be 100 h⁻¹ or less or may be 30 h⁻¹ or less. Here, the WHSV is a ratio of the supply rate (amount supplied /time) F of the raw material gas to the mass W of the dehydrogenation catalyst (F/W) in a continuous reaction apparatus. Note that the amounts of the raw material gas and the catalyst to be used may be appropriately selected in a more preferable range according to reaction conditions and the activity of the catalyst, or the like, and the WHSV is not limited to the range.

In the dehydrogenation step, the reactor may be filled with two or more catalysts.

For example, in the present embodiment, the reactor may be filled with a first dehydrogenation catalyst, which is excellent in catalyst activity of a dehydrogenation reaction from an alkane to an olefin, and a second dehydrogenation catalyst, which is excellent in catalyst activity of a dehydrogenation reaction from the olefin to a conjugated diene. In this aspect, the raw material gas is made to contain an alkane, and a conjugated diene can be efficiently obtained from the alkane.

In the present embodiment, at least one of the first dehydrogenation catalyst and the second dehydrogenation catalyst is required to be the dehydrogenation catalyst mentioned above, and the other may be other dehydrogenation catalyst. Examples of the other dehydrogenation catalyst include noble metal catalysts, catalysts containing Fe and K, and catalysts containing Mo.

Alternatively, in the present embodiment, both the first dehydrogenation catalyst and the second dehydrogenation catalyst may be the dehydrogenation catalyst mentioned above. At this time, either one of the first dehydrogenation catalyst or the second dehydrogenation catalyst may be subjected to a regeneration step mentioned below, or both of these may be subjected to the regeneration step mentioned below.

Next, the regeneration step in the present embodiment will be described in detail

The regeneration step is a step of contacting the dehydrogenation catalyst used in the dehydrogenation step with a regenerating gas containing molecular oxygen under a temperature condition of 310 to 450° C.

The regenerating gas is only required to contain molecular oxygen, and may be, for example, a mixed gas of an oxygen gas and an inert gas (e.g., nitrogen, helium, or argon) or may be air.

The concentration of molecular oxygen in the regenerating gas is not particularly limited, and, for example, may be 0.05% by volume or more, may be 0.1% by volume or more, or may be 0.5% by volume or more. The time required for combustion of coke on the catalyst tends to be shortened by raising the concentration of molecular oxygen in the regenerating gas. Also, the concentration of molecular oxygen in the regenerating gas, for example, may be 20% by volume or less, may be 10% by volume or less, or may be 5% by volume or less.

The temperature condition on regeneration is preferably 310° C. or more, more preferably 330° C. or more. Also, the temperature condition on regeneration may be preferably 450° C. or less, more preferably 430° C. or less.

Coke is deposited on the dehydrogenation catalyst to be subject to the regeneration step. The amount of coke deposited before regeneration, for example, may be 0.1 parts by mass or more or may be 0.5 parts by mass or more, with respect to 100 parts by mass of the dehydrogenation catalyst. Also, the amount of coke deposited before regeneration, for example, may be 20 parts by mass or less or may be 10 parts by mass or less, with respect to 100 parts by mass of the dehydrogenation catalyst.

In the regeneration step, coke deposited on the dehydrogenation catalyst is removed by combustion. The amount of coke deposited after regeneration, for example, is preferably 1.0 part by mass or less, more preferably 0.5 parts by mass or less, particularly preferably 0 parts by mass, with respect to 100 parts by mass of the dehydrogenation catalyst.

The dehydrogenation catalyst after regeneration can be, for example, suitably utilized as a catalyst for a dehydrogenation reaction of an alkane as a raw material or as a catalyst for a dehydrogenation reaction of an olefin as a raw material. The dehydrogenation catalyst after regeneration can be used also as, for example, a catalyst for a dehydrogenation reaction of an oxygen-containing compound such as alcohol, aldehyde, ketone, and carboxylic acid, or a catalyst for a hydrogenation reaction, which is a reverse reaction of the dehydrogenation reaction.

The dehydrogenation catalyst after regeneration may be reused in the dehydrogenation step mentioned above. The dehydrogenation catalyst after regeneration may be also used in steps other than the dehydrogenation step mentioned above.

As described above, a preferred embodiment according to the present invention has been described, but the present invention is not intended to be limited to the embodiment. For example, the present invention has been described above as a method for producing an unsaturated hydrocarbon, but the present invention is not limited to thereto.

One aspect of the present invention may be a regeneration method for regenerating a dehydrogenation catalyst used for a dehydrogenation reaction of a hydrocarbon. This regeneration method may be a method for regenerating a dehydrogenation catalyst by the regeneration step mentioned above.

Another aspect of the present invention may be a method for producing an unsaturated hydrocarbon comprising a dehydrogenation step of performing a dehydrogenation reaction of an alkane using a dehydrogenation catalyst regenerated by the above regeneration method to obtain at least one unsaturated hydrocarbon selected from the group consisting of olefins and conjugated dienes. The dehydrogenation step in this production method may be the same as the dehydrogenation step mentioned above except that the dehydrogenation catalyst after regeneration is used as the dehydrogenation catalyst.

Even another aspect of the present invention may be a method for producing an unsaturated hydrocarbon comprising a dehydrogenation step of performing a dehydrogenation reaction of an olefin using a dehydrogenation catalyst regenerated by the above regeneration method to obtain a conjugated diene. The dehydrogenation step in this production method may be the same as the dehydrogenation step mentioned above except that the dehydrogenation catalyst after regeneration is used as the dehydrogenation catalyst.

### EXAMPLES

Hereinbelow, the present invention will be more specifically described by way of Examples, but the present invention is not intended to be limited to the Examples.

### Example A-1

<Preparation of Dehydrogenation Catalyst A-1>

20.0 g of commercially available γ-alumina (Neobead GB-13, manufactured by Mizusawa Industrial Chemicals, Ltd.) and an aqueous solution prepared by dissolving 25.1 g of magnesium nitrate hexahydrate (Mg(NO₃)₂.6H₂O, manufactured by Wako Pure Chemical Industries, Ltd.) in water (about 150 ml) were mixed, and water was removed in an evaporator at about 50° C. Then, the mixture was dried at 130° C. overnight, fired at 550° C. for 3 hours, and subsequently fired at 800° C. for 3 hours. The obtained fired product and an aqueous solution prepared by dissolving 25.1 g of magnesium nitrate hexahydrate (Mg(NO₃)₂.6H₂O manufactured by Wako Pure Chemical Industries, Ltd.) in water (about 150 ml) were mixed, and water was removed in an evaporator at about 50° C. Then, the mixture was dried at 130° C. overnight, fired at 550° C. for 3 hours, and subsequently fired at 800° C. for 3 hours. Thereby, an alumina-magnesia carrier having a spinel structure was obtained. Note that diffraction peaks derived from the Mg spinel at 2θ=36.9, 44.8, 59.4, and 65.3 deg were observed by X ray diffraction measurement of the obtained alumina-magnesia carrier.

Using a dinitrodiammineplatinum(II) nitric acid solution ([Pt(NH₃)₂(NO₂)₂]/HNO₃ manufactured by TANAKA KIKINZOKU KOGYO K.K.) with respect to 10.0 g of the above alumina-magnesia carrier, impregnation and supporting of platinum were performed in such a way as to achieve an amount of platinum supported of about 1% by mass, and the carrier was dried at 130° C. overnight and fired at 550° C. for 3 hours. Next, the fired product was mixed with an aqueous solution prepared by dissolving 0.62 g of sodium stannate (Na₂SnO₃.3H₂O manufactured by Showa Kako Corp.) in about 30 ml of water, and water was removed in an evaporator at about 50° C. Then, the mixture was dried at 130° C. overnight and fired at 550° C. for 3 hours to thereby obtain a dehydrogenation catalyst A-1.

<Dehydrogenation Reaction Test (1)>

A circulation type reactor of which inner diameter was 10 mmϕ) was filled with 1.0 g of the dehydrogenation catalyst A-1. After hydrogen reduction was performed at 550° C. for 3 hours, a dehydrogenation reaction of butane was perfoinied at a reaction temperature of 550° C. and normal pressure. Butane was used as a raw material, and the raw material gas composition was set to butane:nitrogen:water=1.0:5.3:3.2 (molar ratio). The WHSV was set to 1.0 h⁻¹. Four hours after the reaction was started, each product gas was collected and analyzed by a gas chromatograph (Agilent Technologies, Inc., GC-6850, FID+TCD detector) to determine the butane conversion rate. As a result, the butane conversion rate was 52%.

<Regeneration Test>

A circulation type reactor of which inner diameter was 10 mmϕ was filled with 1.0 g of the dehydrogenation catalyst A-1. After hydrogen reduction was performed at 450° C. for 40 minutes, 1-butene, at 4.5 g/h, and nitrogen, at 20 cc/min, were fed at a reaction temperature of 570° C. under normal pressure for 30 minutes. Then, after switchover to nitrogen and temperature lowering to 450° C., regeneration was performed by supplying a mixed gas of air and nitrogen for 90 minutes. The amount of coke on the dehydrogenation catalyst A-1, after this operation was repeated 5 times, was measured with a thermogravimetric balance. Note that the ratio of the reduced weight under air (w) to the weight (W), after the catalyst was retained under nitrogen at 300° C. for 10 minutes, the temperature was raised to 600° C., and the catalyst was retained for 10 minutes, using a thermogravimetric balance, was taken as the amount of coke (%). The amount of coke on the dehydrogenation catalyst after regeneration A-1 was 0%.

<Dehydrogenation Reaction Test (2)>

0.9 g of the dehydrogenation catalyst A-1 after regeneration was taken, and a circulation type reactor of which inner diameter was 10 mmϕ) was filled with the catalyst A-1. After hydrogen reduction was performed at 550° C. for 3 hours, a dehydrogenation reaction of butane was performed at a reaction temperature of 550° C. and normal pressure. Butane was used as a raw material, and the raw material gas composition was set to butane:nitrogen:water=1.0:5.3:3.2 (molar ratio). The WHSV was set to 1.0 Four hours after the reaction was started, each product gas was collected and analyzed by a gas chromatograph (Agilent Technologies, Inc., GC-6850, FID+TCD detector) to determine the butane conversion rate. As a result, the butane conversion rate was 43%. Additionally, the ratio of the butane conversion rate in the dehydrogenation reaction test (2) to the butane conversion rate in the dehydrogenation reaction test (1) was 0.83.

### Example A-2

The regeneration test and the dehydrogenation reaction test (2) were performed in the same manner as in Example A-1 except that the regeneration temperature in the regeneration test was changed to 400° C. The butane conversion rate in the dehydrogenation reaction test (2) was 52%, and the ratio to the butane conversion rate in the dehydrogenation reaction test (1) was 1.0.

### Comparative Example a-1

The regeneration test and the dehydrogenation reaction test (2) were performed in the same manner as in Example A-1 except that the regeneration temperature in the regeneration test was changed to 550° C. The butane conversion rate in the dehydrogenation reaction test (2) was 26%, and the ratio to the butane conversion rate in the dehydrogenation reaction test (1) was 0.50.

### Comparative Example a-2

The regeneration test and the dehydrogenation reaction test (2) were performed in the same manner as in Example A-1 except that the regeneration temperature in the regeneration test was changed to 500° C. The butane conversion rate in the dehydrogenation reaction test (2) was 34%, and the ratio to the butane conversion rate in the dehydrogenation reaction test (1) was 0.65.

### Comparative Example a-3

The regeneration test was performed in the same manner as in Example A-1 except that the regeneration temperature in the regeneration test was changed to 300° C., but deposition of coke on the dehydrogenation catalyst was observed, and it was not possible to sufficiently remove coke.

### Example B-1

<Preparation of Dehydrogenation Catalyst B-1>

10.0 g of commercially available γ-alumina (Neobead GB-13, manufactured by Mizusawa Industrial Chemicals, Ltd.) and an aqueous solution prepared by dissolving 1.65 g of sodium stannate (Na₂SnO₃.3H₂O manufactured by Showa Kako Corp.) in about 50 ml of water were mixed, and water was removed in an evaporator at about 50° C. Then, the mixture was dried at 130° C. overnight and fired at 550° C. for 3 hours. Next, using an aqueous solution of tetraammineplatinum(II) nitrate ([Pt(NH₃)₄](NO₃)₂ manufactured by TANAKA KIKINZOKU KOGYO K.K.), impregnation and supporting of platinum were performed in such a way as to achieve an amount of platinum supported of about 1% by mass, and the carrier was dried at 130° C. overnight and fired at 550° C. for 3 hours to thereby obtain a dehydrogenation catalyst B-1.

<Dehydrogenation Reaction Test (1)>

A circulation type reactor of which inner diameter was 10 mmϕ was filled with 1.0 g of the dehydrogenation catalyst B-1. After hydrogen reduction was performed at 550° C. for 3 hours, a dehydrogenation reaction of butane was performed at a reaction temperature of 550° C. and normal pressure. Butane was used as a raw material, and the raw material gas composition was set to butane:nitrogen:water=1.0:5.3:3.2 (molar ratio). The WHSV was set to 1.0 h⁻¹. Four hours after the reaction was started, each product gas was collected and analyzed by a gas chromatograph (Agilent Technologies, Inc., GC-6850, FID+TCD detector) to determine the butane conversion rate. As a result, the butane conversion rate was 59%.

<Regeneration Test>

A circulation type reactor of which inner diameter was 10 mmϕ was filled with 1.0 g of the dehydrogenation catalyst B-1. After hydrogen reduction was performed at 450° C. for 40 minutes, 1-butene, at 4.5 g/h, and nitrogen, at 20 cc/min, were fed at a reaction temperature of 570° C. under normal pressure for 30 minutes. Then, after switchover to nitrogen and temperature lowering to 400° C., regeneration was performed by supplying a mixed gas of air and nitrogen for 90 minutes. When the amount of coke on the dehydrogenation catalyst B-1, after this operation was repeated 5 times, was measured with a thermogravimetric balance, the amount of coke was 0%.

<Dehydrogenation Reaction Test (2)>

0.9 g of the dehydrogenation catalyst B-1 after regeneration was taken, and a circulation type reactor of which inner diameter was 10 mmϕ was filled with the catalyst. After hydrogen reduction was performed at 550° C. for 3 hours, a dehydrogenation reaction of butane was performed at a reaction temperature of 550° C. and normal pressure. Butane was used as a raw material, and the raw material gas composition was set to butane:nitrogen:water=1.0:5.3:3.2 (molar ratio). The WHSV was set to 1.0 h⁻¹. Four hours after the reaction was started, each product gas was collected and analyzed by a gas chromatograph (Agilent Technologies, Inc., GC-6850, FID+TCD detector) to determine the butane conversion rate. As a result, the butane conversion rate was 48%. Additionally, the ratio of the butane conversion rate in the dehydrogenation reaction test (2) to the butane conversion rate in the dehydrogenation reaction test (1) was 0.81.

### Example C-1

<Preparation of Dehydrogenation Catalyst C-1>

An alumina-magnesia carrier was prepared in the same manner as in Example A-1. To 3.0 g of the obtained alumina-magnesia carrier, an aqueous solution prepared by dissolving 79.6 mg of H₂PtCl₆.2H₂O in 16 mL of water was added. The obtained mixed solution was stirred at 40° C. and 0.015 MPaA for 30 minutes and at 40° C. and normal pressure for 30 minutes, using a rotary evaporator. Then, water was removed under reduced pressure while the mixed solution was stirred. The obtained solid was dried in an oven at 130° C. overnight. Next, the dried solid was fired under an air flow at 550° C. for 3 hours. To the obtained solid, a solution prepared by dissolving 0.311 g of SnCl₂.2H₂O in 20 ml, of EtOH was added. The obtained mixed solution was stirred at 40° C. under normal pressure for an hour using a rotary evaporator, and then, EtOH was removed under reduced pressure. The obtained solid was dried in an oven at 130° C. overnight. Next, the dried solid was fired under an air flow at 550° C. for 3 hours. Hydrogen reduction was further performed at 550° C. for 2 hours to thereby obtain a dehydrogenation catalyst C-1.

<Dehydrogenation Reaction Test (1)>

A circulation type reactor of which inner diameter was 10 min+was filled with 1.0 g of the dehydrogenation catalyst C-1. After hydrogen reduction was performed at 550° C. for 3 hours, a dehydrogenation reaction of butane was performed at a reaction temperature of 550° C. and normal pressure. Butane was used as a raw material, and the raw material gas composition was set to butane:nitrogen:water=1.0:5.3:3.2 (molar ratio). The WHSV was set to 1.0 h⁻¹.Four hours after the reaction was started, each product gas was collected and analyzed by a gas chromatograph (Agilent Technologies, Inc., GC-6850, FID+TCD detector) to determine the butane conversion rate. As a result, the butane conversion rate was 60%.

<Regeneration Test>

A circulation type reactor of which inner diameter was 10 mmϕ was filled with 1.0 g of the dehydrogenation catalyst C-1. After hydrogen reduction was performed at 450° C. for 40 minutes, 1-butene, at 4.5 g/h, and nitrogen, at 20 cc/min, were fed at a reaction temperature of 570° C. under normal pressure for 30 minutes. Then, after switchover to nitrogen and temperature lowering to 400° C., regeneration was performed by supplying a mixed gas of air and nitrogen for 90 minutes. When the amount of coke on the dehydrogenation catalyst C-1, after this operation was repeated 5 times, was measured with a thermogravimetric balance, the amount of coke was 0%.

<Dehydrogenation Reaction Test (2)>

0.9 g of the dehydrogenation catalyst C-1 after regeneration was taken, and a circulation type reactor of which inner diameter was 10 mmϕ was filled with the catalyst. After hydrogen reduction was performed at 550° C. for 3 hours, a dehydrogenation reaction of butane was performed at a reaction temperature of 550° C. and normal pressure. Butane was used as a raw material, and the raw material gas composition was set to butane:nitrogen:water=1.0:5.3:3.2 (molar ratio). The WHSV was set to 1.0 h⁻¹. Four hours after the reaction was started, each product gas was collected and analyzed by a gas chromatograph (Agilent Technologies, Inc., GC-6850, FID+TCD detector) to determine the butane conversion rate. As a result, the butane conversion rate was 39%. Additionally, the ratio of the butane conversion rate in the dehydrogenation reaction test (2) to the butane conversion rate in the dehydrogenation reaction test (1) was 0.65.

### Example D-1

<Preparation of Dehydrogenation Catalyst D-1>

An alumina-magnesia carrier was prepared in the same manner as in Example A-1. With respect to 10.0 g of the obtained alumina-magnesia carrier, an aqueous solution prepared by dissolving 3.7 g of sodium stannate (Na,SnO₃.3H₂O manufactured by Showa Kako Corp.) in about 100 ml of water, and water was removed in an evaporator at about 50° C. Then, the mixture was dried at 130° C. overnight and fired at 550° C. for 3 hours. Next, using an nitric acid solution of dinitrodiammineplatinum(II) ([Pt(NH₃)₂(NO₂)₂]/HNO₃ manufactured by TANAKA KIKINZOKU KOGYO K.K.), impregnation and supporting of platinum were performed in such a way as to achieve an amount of platinum supported of about 1% by mass, the carrier was dried at 130° C. overnight, and fired at 550° C. for 3 hours to thereby obtain a dehydrogenation catalyst D-1.

<Dehydrogenation Reaction Test (1)>

A circulation type reactor of which inner diameter was 10 mmϕ was filled with 1.0 g of the dehydrogenation catalyst D-1. After hydrogen reduction was performed at 550° C. for 3 hours, a dehydrogenation reaction of butene was performed at a reaction temperature of 600° C. under normal pressure. 2-butene (a mixture of trans-2-butene and cis-2-butene) was used as a raw material, and the raw material gas composition was set to 2-butene:nitrogen:water=1.0:5.3:3.2 (molar ratio). The WHSV was set to 1.0 h⁻¹. Four hours after the reaction was started, each product gas was collected and analyzed by a gas chromatograph (Agilent Technologies, Inc., GC-6850, FID+TCD detector) to determine the butene conversion rate. As a result, the butene conversion rate was 38%.

<Regeneration Test>

A circulation type reactor of which inner diameter was 10 mmϕ was filled with 1.0 g of the dehydrogenation catalyst D-1. After hydrogen reduction was performed at 450° C. for 40 minutes, 1-butene, at 4.5 g/h, was fed at a reaction temperature of 600° C. under normal pressure for 60 minutes. Then, after switchover to nitrogen and temperature lowering to 400° C., regeneration was performed by supplying a mixed gas of air and nitrogen for 90 minutes. When the amount of coke on the dehydrogenation catalyst D-1, after this operation was repeated 5 times, was measured with a thermogravimetric balance, the amount of coke was 0%.

<Dehydrogenation Reaction Test (2)>

0.9 g of the dehydrogenation catalyst D-1 after regeneration was taken, and a circulation type reactor of which inner diameter was 10 mmϕ was filled with the catalyst. After hydrogen reduction was performed at 550° C. for 3 hours, a dehydrogenation reaction of butene was performed at a reaction temperature of 600° C. and normal pressure. 2-butene (a mixture of trans-2-butene and cis-2-butene) was used as a raw material, and the raw material gas composition was set to 2-butene:nitrogen:water=1.0:5.3:3.2 (molar ratio). The WHSV was set to 1.0 h⁻¹. Four hours after the reaction was started, each product gas was collected and analyzed by a gas chromatograph (Agilent Technologies, Inc., GC-6850, FID+TCD detector) to determine the butene conversion rate. As a result, the butane conversion rate was 38%. Additionally, the ratio of the butane conversion rate in the dehydrogenation reaction test (2) to the butane conversion rate in the dehydrogenation reaction test (1) was 1.0.

### INDUSTRIAL APPLICABILITY

According to the method for regenerating a dehydrogenation catalyst in accordance with the present invention, it is possible to efficiently remove coke deposited on the dehydrogenation catalyst while sufficiently maintaining the catalyst activity of the dehydrogenation catalyst. Additionally, according to a method for producing an unsaturated hydrocarbon in accordance with the present invention, it is possible to achieve efficiency improvement of the entire process by efficiently removing coke deposited on the dehydrogenation catalyst while sufficiently maintaining the catalyst activity of the dehydrogenation catalyst.

1. A method for producing an unsaturated hydrocarbon comprising:
a dehydrogenation step of contacting a raw material gas containing at least one hydrocarbon selected from a group consisting of alkanes and olefins with a dehydrogenation catalyst containing a group 14 metal element and Pt to obtain a product gas containing at least one unsaturated hydrocarbon selected from a group consisting of olefins and conjugated dienes, and
a regeneration step of contacting the dehydrogenation catalyst subjected to the dehydrogenation step with a regenerating gas containing molecular oxygen under a temperature condition of 310 to 450° C.

2. The method according to claim 1, wherein the group 14 metal element includes Sn.

3. The method according to claim 1, wherein the dehydrogenation catalyst is a catalyst in which a group 14 metal element and Pt are supported on a carrier using a metal source containing no chlorine atom.

4. The method according to claim 1, wherein the raw material gas contains an alkane having 2 to 10 carbon atoms.

5. The method according to claim 1, wherein the raw material gas contains an olefin having 4 to 10 carbon atoms.

6. A method of regenerating a dehydrogenation catalyst containing a group 14 metal element and Pt that has been used for a dehydrogenation reaction of a hydrocarbon, the method comprising:
a regeneration step of contacting the dehydrogenation catalyst with a regenerating gas containing molecular oxygen under a temperature condition of 310 to 450° C.

7. A method for producing an unsaturated hydrocarbon comprising:
a step of performing a dehydrogenation reaction of an alkane using a dehydrogenation catalyst regenerated by the regeneration method according to claim 6 to obtain at least one unsaturated hydrocarbon selected from the group consisting of olefins and conjugated dienes.

8. A method for producing an unsaturated hydrocarbon comprising a step of performing a dehydrogenation reaction of an olefin using a dehydrogenation catalyst regenerated by the regeneration method according to claim 6 to obtain a conjugated diene.
